(19) United States
(12) Patent Application Publication
(10) Pub. No.: US 2006/0062837 A1
(43) Pub. Date: Mar. 23, 2006

(54) Dual activating TDD

(76) Inventor: Roger Timothy Putman SR., Collinsville, IL (US)

Correspondence Address: Roger Timothy Putman, Sr, 300 Clarence Street, Collinsville, IL, 62234-1013, US

(21) Appl. No.: 10/945,658

(22) Filed: Sep. 21, 2004

(51) Int. Cl. A61K 9/70

(52) U.S. Cl. 424/449

(57) ABSTRACT

Preferred embodiment FIG. 1, comprises a novel transdermal delivery device for ensuring longer shelf life of unstable drugs comprising a patch for disbursement of therapeutic drugs to the skin of a user; said patch comprising an inbuilt injection port in which the user injects the diluent and/or active ingredient(s) into the drug reservoir to activate said drug delivery device, wherein the device comprises various permeablity rate-controlling membranes to accommodate a specific drug being introduced to the device, wherein upon removing the protective outer layer, of the preferred embodiment FIG. 1. the device is ready to be placed on the skin by the patient. Additional embodiment replaces the injection port with a separate sealed diluent pouch reservoir.

This application claims benefit of the Provisional Appl. No. 60/503,662 filed; Sep. 19^{th} 2003.

### STATEMENT REGARDING FEDERALLY SPONSORED RESEARCH OR DEVELOPMENT

“Not Applicable”

### REFERENCE TO A SEQUENCE LISTING

Sequence listing included herewith, separately.

### BACKGROUND FIELD OF THE INVENTION

The invention outlined in this application is related to an improved and novel transdermal drug delivery device, abbreviated as, TDD. More specifically, the invention is a transdermal delivery device for ensuring longer shelf life of an unstable drug and made ready by the end user.

As examples of various types of transdermal drug delivery devices that have been developed, reference is made to U.S. Pat. Nos. listed below;

| | | |
|---|---|---|
| 2002/0168401 | Pub. Date: Nov. 14, 2002 | Kanios et al. |
| 2001/0051180 A1 | Pub. Date: Dec. 13, 2001 | Watanabe et al. |
| 2001/0048987 | Pub. Date: Dec. 6, 2001 | David Kanios |
| 5,706,937 | Pub. Date: Jan. 13, 1998 | Futagawa et al. |
| 5,662,925 | Pub. Date: Sep. 2, 1997 | Ebert et al. |
| 5,614,211 | Pub. Date: Mar. 25, 1997 | Gale et al. |

### BACKGROUND OF THE INVENTION

Originally, conventional transdermal drug delivery devices of the art are flexible and have a partition dividing the interior of the device.

The layers of the device are introduced to the diluent by breaking a partition.

One disadvantage of these systems is that a small amount of diluent and/or gases can permeate across the partition, which are generally made of a synthetic resin.

Secondly, problems of the known transdermal drug delivery devices available on the market today is that frequently, the permeating form of the drug is not suitably stable; therefore, the shelf life of the system would be too short to be commercially practical.

Additionally, a problem encountered by the known transdermal drug delivery devices is the problem of “drug leakage”, primarily through the partician and adhesive. It can redistribute itself from the reservoir into the adhesive, and if the adhesive (and permeable membrane) have edges which are not surrounded by an occlusive covering, drug loss results.

In the case where a hygroscopic and unstable drug such as an antibiotic is held in one reservoir, and a diluent is held in the other reservoir, even a small “inter-reservoir” permeation of diluent or gases are problematic.

Another problem of the known transdermal drug delivery devices available on the market today is that frequently, the permeating form of the drug is not suitably stable; therefore, the shelf life of the device would be too short to be commercially practical.

In addition, transdermal systems of the art are limited to regulating drugs delivery by only a few, very limited means; i.e. drug concentration, membrane or matrix material and thickness, and fulx enhancers.

It is an object of the present invention to provide a transdermal drug delivery device that is more stable than the systems currently available.

Another objective of this device is to provide a mechanism to store the diluents and active ingredient(s) under different storage conditions.

Another object of this invention to provide a transdermal device that will allow the end user to be able to mix the diluent and the active ingredient(s) prior to use with minimal training.

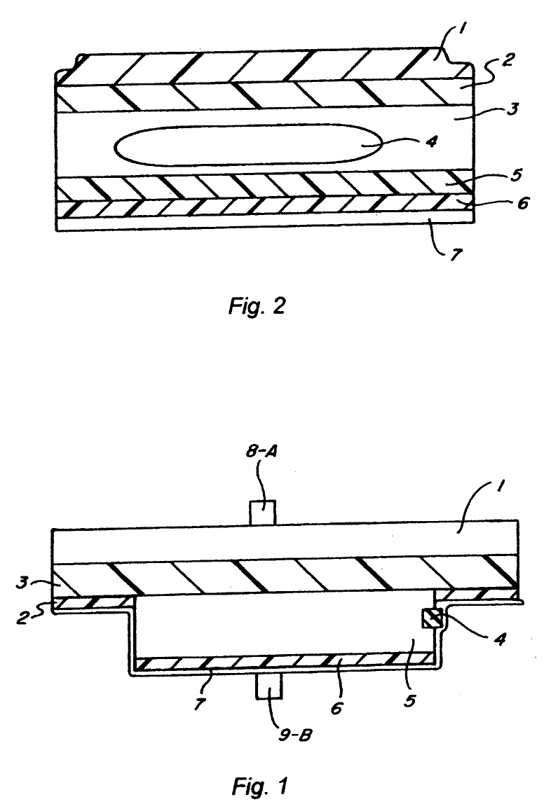

It is an additional object of the invention to provide a transdermal drug delivery device that allows the user or health professional to use different amounts of diluent and/or active ingredient(s) based on the need of a particular patient. These objects are possible in FIG. 1, the preferred embodiment, in which the diluent and/or active ingredient(s) are injected prior to using the device.

Another object of the invention is to allow the manufacture of the transdermal delivery components separately, under separate manufacturing conditions that may be unique to each component.

Yet another object of the present invention is to allow the healthcare professional to prescribe different diluents and/or active ingredient(s) to be injected into the device depending on the need of particular patients.

The above listed objectives, and many other benefits of the new invention are achieved by having a transdermal drug delivery device that comprises a reservoir for the active ingredient(s) by itself, or in combination with other inert ingredients to enhance its volume, stability or both.

In addition, within the same reservoir of embodiment FIG. 2, contains another separate sealed reservoir containing the diluent, which is in the liquid form.

This additional reservoir can be ruptured by applying a force preferably between 10-25 pounds, at a particular point to allow the leakage and subsequent mixing of the diluent with the active ingredient(s).

Alternatively and preferably, the system comprises a self-sealable port of a suitable flexible material through which a thin needle can be passed to inject the diluent and/or active ingredient(s) into the reservoir.

The volume and the composition of the diluent and/or active ingredient(s) can vary to allow flexibility in the delivery characteristics.

This is an unique advantage that is not available in any of the available devices currently on the market. Upon mixing the active ingredient(s) and diluent, the device is ready to use by the patient.

The device may be used for extended periods of time, or the diluent may be formulated to allow the drug delivery for a period of 4 to 24 hours or longer. The length of the drug delivery may also be varied by varying either the composition of the diluent, or the volume of the active ingredient(s) injected.

Another advantage of the present invention is that it can be used for active ingredient(s) that are less stable in the presence of diluents, and/or react with the diluents over an extended period of time.

Mixing of the active ingredient(s) and diluent is achieved by either injecting the diluent and/or active ingredient(s) into the system with the help of an inbuilt injection port of the preferred embodiment of FIG. 1, or by rupturing a separate sealed reservoir containing the diluent, of FIG. 2.

Still further objects and advantages will become apparent from a consideration of the ensuing description and drawings.

### BRIEF SUMMARY OF THE INVENTION

In accordance with the invention embodiments outlined within these pages, its possible to administer numerous certain drugs previously thought to be too unstable or practical for commercial use. It is the essence of this invention outlined herein, to allow these numerous unstable drug categories to be useful in transdermal drug delivery therapy.

### BRIEF DESCRIPTION OF THE DRAWINGS

Preferred Embodiment FIG. 1 comprising;
(a) outermost removable layer number 1;
(b) layer 2 is an adhesive layer;
(c) layer 3 is attached to an adhesive layer on the outside periphery of layer 2 to allow adhesion to the skin;
(d) component 4 is an injection port;
(e) layer 5 is an active ingredient reservoir;
(f) layer 6 is an adhesive layer
(g) layer 7 is the peel-off protective layer;
(h) tab A is attached to layer 1;
(i) tab B is attached to layer 7;

EMBODIMENT FIG. 2 comprising;
(a) outer layer 1, being the protective outer layer;
(b) layer number 2 is a fluid impermeable outer membrane to hold contents of active therapeutic agent substance reservoir containing a therapeutic substance agent without allowing it to leak out for the topside;
(c) layer number 3 is the active ingredient(s) layer comprising a membrane containing the dissolved active ingredient(s) or simply a layer of the solid active ingredient(s) and any inert materials associated with it and may contain an open space that can accommodate a sealed diluent agent pouch;
(d) numbered 4 in the device is the separate sealed diluent pouch reservoir;
(e) number 5 being the rate-controlling permeable membrane;
(f) number 6 being the adhesive layer;
(g) layer 7 being the peel-off protective layer;

### DETAILED DESCRIPTION OF THE INVENTION

The preferred embodiment FIG. 1, comprises;

The device is prepared by starting with the outermost layer number 1. This layer is a removable layer and is comprised of suitable materials such as silanized plastic or polyester film, or metalized polyester film. The film is usually about 250-500 microns thick. The reason for this higher thickness is that the device needs some degree of rigidity in order to allow the injection of diluent and/or active ingredient(s) without comprising the membranes around the injection port.

The layers may be attached together with the help of an adhesive that can be easily pealed to allow the removal of layer number 1 from the device.

Layer 2 is an adhesive layer with no permeability properties.

Layer 2 and 3 are attached only at the periphery, with the area of attachment being 0.5-2 cm all around the device.

The material of the layer number 3 is typically a translucent polyolefin that is ideal for elastic films that do not rupture easily. It may also consist of a polyester film providing high strength and barrier properties that are essential for this device to withstand the handling and application stresses.

The material of the layer number 3 is typically a translucent polyolefin that is ideal for elastic films that do not rupture easily. It may also consist of a polyester film providing high strength and barrier properties that are essential for this device to withstand the handling and applications stresses.

Additionally, this layer has to serve as the barrier to keep the drug solution from leaking out from the outside of the device.

Layer 3 is attached to an adhesive layer, on the outside periphery to allow adhesion to the skin.

This peripheral area extends beyond the remaining all layers of the system. The reason for this extension is that once an injection is made in the device to mix the active ingredient(s) and diluent, the injection port is covered by the adhesive that attaches to the skin.

Such a design allows the device to effectively hide the injection port after the device has been activated so that the injection port, which is relatively structure, will not get caught in the clothing or any other object after application.

The extended areas of the layers 2 and 3 will completely cover it and will also serve to attach the system to the skin. The material for the adhesive layer is a polymer such as polyisobutylene, or paper substrate with silicon coating.

This layer is firmly attached to layer number 3 because it will stay with the device throughout the use, and does not have to be peeled off. It may be attached to layer 3, by heat and pressure combination, or simply by a suitable adhesive of permanent nature.

The layer number 5 is the active ingredient(s) reservoir and consists of a mixture of the drug to be delivered along with some inert ingredients that may be required to build up the bulk of the active ingredient or improve its stability.

The entire ingredient(s) mixture is usually in the form of a solid mixture, or can be in the form of a solid layer deposited on the layer number 3 or 6.

Alternatively, for certain drugs, the active ingredient(s) mixture can be in a fluid, semi-fluid or gel state as well to allow the drug to remain dissolved in the system.

The final state of the active ingredient is dictated by the nature of the active ingredient.

More stable and poorly soluble actives could be in the solution form, whereas less stable actives such as polypeptides could be in the solid form. As mentioned earlier, the solid form can simply be a powder enclosed between the layers 3 and 6, or a layer of material deposited on layers 3 or 6 or both.

Component 4 of the system is an injection port that substitutes for the diluent pouch in embodiment 2.

This port is made of materials that allow passage of a thin needle of gauge 25-30 and has the elasticity to allow seal of the hole after the needle is withdrawn.

This injection port is connected to the system, being attached between layers 3 and 6 on one side of the system.

The thickness of this port ranges between 1-3 mm, preferably about 2 mm to allow the needle to be inserted and the diluent and/or active ingredient(s) injected. Since this area will be fully covered by the adhesive layer (layer 2) that extends beyond the system by 1-2 cm, the injection port will not be visible after the diluent and/or active ingredient(s) have been added and attached to the skin.

The materials suitable for injection port are siliconized rubber or similar components that have the property of self-sealing. The port is attached to the layers by using a suitable adhesive between the port material and the layers.

For a better design, the injection port may have varying thickness and attached to a plastic tubing as a guide to insure non-perforation of the device, being thickest in the middle, and getting thinner on the sides that form a seal with the membranes.

This will allow the membranes to seal effectively and firmly around the injection port.

The depth to which goes into the drug reservoir is typically 2-4 mm, preferably about 2 mm.

Layer number 6 is the rate-controlling layer for the device. This layer consists of a suitable material that is flexible and has suitable permeability.

The degree of permeability desired varies with the nature of active ingredient(s).

For polypeptides, the membrane number 6 should be highly permeable, essentially offering no resistance to the material flow, whereas for smaller organic molecules such as steroids, the membrane can have varying degree of resistance to the drug permeation.

This layer, also sometimes known as the release liner is typically made of materials such as polyisobytylene, acrylic or silicone membranes or a paper substrate liner with silicone coating. It can also be made up of polyester and polyurethane liners or low-density polyethylene films with or without a fluorosilicone coating on either side.

Typical thickness of this membrane is 50-100 microns. The layer number 6 of the device is also the adhesive layer that attaches the patch to the patient's skin.

One requirement of layer 6 is that it should be able to withstand the pull force that will be applied during the process of injection. It should also be resistant to tearing and shear due to possibility of injection needle coming in contact with it. The mechanism of injection, as explained in paragraph 0055 the possibility of this contact.

The last layer, number 7, in the system is the peel off protective layer. The function of this layer is to protect the adhesive surface of the system. This layer is attached to the adhesive layer simply by sticking to the surface, and can be easily peeled off to expose the adhesive surface.

The material for this layer is the same as for layer number 1. It can be either silanized polyester or metalized polyester.

The thickness varies between 50 to 200 microns.

The entire system can be prepared by either heat-sealing various layers with each other, or by use of suitable non-toxic adhesives.

Volume of the diluent to be injected varies from 50 to 200 ul. The diluent may contain a mixture of ingredients such as the solvent including water and alcohol, permeability and/or flux enhancing agents, stabilizers, buffers, pH altering ingredients, complexing agents, gelling or swelling agents, etc. These materials are in a free flowing state so that upon rupture of the system, the material is quickly released into the drug reservoir, and mixes efficiently to form a uniform mixture. The final form of this mixture can be a solution, gel, cream, lotion or any other suitable form. Additionally, the diluent may contain ingredients to form special delivery systems such as microemulsions, liposomes, etc.

The device contains two tabs, A and B, numbered as 8,9. Tab A is attached to layer 1 and Tab B is attached to layer 7. The purpose of these tabs is two-fold. First, these allow the layers to be peeled off before the system is applied on the skin. Secondly, the tabs will allow the system to be held open while the injection is being made into the active reservoir. This will minimize the chances of rupturing the membranes 2 or 6 during the process of injection.

The method of injection involves simply pulling the tabs to slightly expand the device. Once the device is expanded the needle attached to a syringe containing the diluent and/or active ingredient(s) is inserted into the injection port, and injected into the active reservoir. Once the injection process is complete, the needle is withdrawn, the tabs pulled with a higher force to remove the layers 1 and 7 and the system is applied on the skin.

Once the system is in place on the skin, it can be slightly pressed on all sides to make the drug solution uniform across the system.

Relatively higher thickness of layers (50-200 microns) allows the layers to hold the rest of the system in place and allow the system to expand to allow easy injection through the injection port.

Rest of the layers together make up about 200 micron thickness, with the injection port area having a total thickness of about 2-3 mm. Since the ends of the injection port are tapered, there is no leak from the sides of the port.

The extended part of adhesive layer 3 fully covers the injection port after the system is in place on the skin.

As a person of ordinary skill is aware, several modifications of the above embodiments are possible. One such variation can have the layer 2 in embodiment 1 contain a middle seal of resealable rubber, making it possible to inject the material from the top of the system. This will also result in a thinner overall system, but may slightly increase the chances of puncturing a membrane in the process of injection.

FIG. 2, represents an additional embodiment described below;

Typically, the device is prepared by starting with the outermost layer number 1. This is the membrane that is handled by the patient and is used to protect the system.

This layer is a removable layer and is comprised of suitable material such as silanized plastic or polyester film. The film is usually about 50-100 micron thick. The layers may be attached together with the help of an adhesive that can be easily pealed to allow the removal of layer number 1 from the system. It may also be left in place and used to remove the system from the skin at the end of the treatment period.

The material of the layer number 2 is typically an translucent polyolefin that is ideal for elastic films that do not rupture easily. It may also consist of a polyester film providing high strength and barrier properties that are essential for this system to withstand the force of rupturing the diluent pouch.

Layer number 3 is the active ingredient(s) reservoir and consists of a mixture of the drug or drugs to be delivered along with some inert ingredients that may be required to build up the bulk of the active ingredient(s) or improve its stability. The entire active ingredient(s) mixture is usually in the form of a solid mixture, or can be in the form of a solid layer deposited on the layer number 2 or 5. Alternatively, for certain drugs, the active ingredient(s) mixture can be in a fluid, semi-fluid or gel state as well to allow the drug to remain dissolved in the device.

The final state of the active ingredient is dictated by the nature of the active ingredient.

More stable and poorly soluble actives could be in the solution form, whereas less stable actives such as polypeptides could be in the solid form. As mentioned earlier, the solid form can simple be a powder enclosed between the layers 2 and 5, or a layer of material deposited on layers 2 or 5 or both.

Component 4 of the device is a pouch that contains the diluent. This pouch is not attached to the rest of the system, thus imparting it the ability to have independent properties.

The materials for the pouch can be any plastic material of thickness ranging from 25-75 microns. The suitable materials will be the membranes with low tensile strength to allow the pouch to rupture upon application of pressure of preferably 10 to 30 pounds. It can be made of low-density acrylic films. The pouch is typically filled to capacity to not allow any room for expansion, thus making it easy to rupture the pouch. The about 25 pounds being the preferred amount of pressure to rupture the pouch. Too low a rupture pressure will allow the device to be activated prematurely, whereas high-pressure pouch rupture will make it hard to activate the device.

Layer number 5 is the rate-controlling layer for the device. This layer consists of a suitable material that is flexible to withstand the rupture pressure, and has suitable permeability.

For polypeptides, the membrane number 5 should be highly permeable, essentially offering no resistance to the material flow, whereas for smaller organic molecules such as steroids, the membrane can have varying degree of resistance to the drug permeation.

In all cases, however, the permeability of this layer cannot be high enough to allow seepage of the solution across it, resulting in loss of the drug solution that will result after the pouch is ruptured. This layer, also sometimes know as the release liner is typically made of materials such as polyisobytylene, acrylic or silicone membranes or a paper substrate liner with silicone coating.

It can also be made up of polyester and polyurethane liners or low-density polyethlene films with or without a fluorosilicone coating on either side. Typical thickness of this membrane is 50-100 microns.

The layer number 6 of the device is the adhesive layer that attaches the device to the patient's skin. This membrane is made up of essentially the same material as layer number 5 but usually does not have any barrier function for the drug release. Its sole purpose may be to allow the device to stick to the skin for a desired period of time, such as 4 to 24 hours or longer, and allow for removal of the device without hurting the skin. In some devices, this layer may be combined with layer number 5 to serve both as the rate-controlling and adhesive layers.

The layer number 6 of the device is the adhesive layer that attaches the device to the patient's skin. This membrane is made up of essentially the same material as layer number 5 but usually does not have any barrier function for the drug release. Its sole purpose may be to allow the device to stick to the skin for a desired period of time, such as 4 to 24 hours or longer, and allow for removal of the device without hurting the skin. In some devices, this layer may be combined with layer number 5 to serve both as the rate-controlling and adhesive layers.

Many commercially available films such as polyisobutylene and polyester films can be used for this purpose. The thickness of this film is in the same range as that for the layer number 5, i.e., 50-100 microns.

One example of the commercially available adhesive films that can also serve as permeability controlling films is the Mediderm series of films from Mylan Technologies Inc. There are many other suppliers for similar films.

One of the requirements for layer 6 is that it should be able to withstand the rupture pressure of the diluent pouch. Most of the materials used for this film such as polyisobytylene and polyester films have the strength to withstand such a pressure.

The user can be trained to place the device on a flat surface before applying pressure from the top, in which case only the pouch is subject to pressure.

It is possible to do away with one of the number 5 or 6 layers because the two functions of the permeability layer and adhesive can be combined in one layer.

The two layers are proposed in the system to allow for the flexibility of design to accommodate needs for various active ingredients.

The last layer number 7 in the device is the peel off protective layer. The function of this layer is to protect the adhesive surface of the system. This layer is attached to the adhesive layer simply by sticking to the surface, and can be easily peeled off to expose the adhesive surface. The material for this layer is the same as for layer number 1.

It can be either silanized polyester or metalized polyester. The thickness varies between 25 to 50 microns.

The entire device is enclosed in a plastic film to protect it during handling and shipping. The plastic film that wraps around the entire device is expected to be of enough strength to protect the device from pressure that is enough to rupture the diluent reservoir, causing premature mixing of the diluent with active ingredient(s).

### CONCLUSION, RAMIFICATIONS, AND SCOPE

Accordingly, the reader will find that according to the invention set forth in this patent application; I have provided within the specifications and detailed description, an improved means for preparing a useful and efficient two embodiment transdermal drug delivery device, in which to deliver a wide range of medicaments such as, but not limited to; cardiovascular, peptides, polypeptides, small organic, steroids, and vitamins, some of which were previously thought to be unable to be effectively delivered transdermally. The method of activation and storage capabilities for both embodiments are thought to be an improvement in the known art.

What we claim is;

1. A novel transdermal delivery device for ensuring longer shelf life of unstable drugs comprising a patch for disbursement of therapeutic drugs to the skin of a user; said patch comprising an inbuilt injection port in which the user injects the diluent and/or active ingredient(s) into the drug reservoir to activate said drug delivery device, wherein the device has a varied permeable rate-controlling membrane to accommodate the specific drug being introduced to the device, wherein upon removing the protective outer layer, of the preferred embodiment FIG. 1. the device is ready to be placed on the skin by the patient.

2. Additional Embodiment of the present invention comprising; said patch including a separate sealed pouch component comprising a liquid diluent within the active ingredient(s) reservoir, represented in embodiment 2 of the present invention; wherein the user must apply between 10-30 pounds of pressure to burst said diluent pouch to mix the active ingredient(s), wherein said active ingredient(s) migrate through a permeable rate-controlling membrane to the surface of the patch, wherein the user must remove the protective outer layer before attaching said patch to the skin.

3. A transdermal delivery device of claim 1, to administer medicaments such as, but not limited to; cardiovascular, peptides, polypeptides, small organic, protein, steroids, vitamins;

4. A method of claim 2 wherein; the mixing of the diluent and/or active ingredient(s) by injecting the diluent and/or active ingredient(s) through the inbuilt injection port of the preferred embodiment FIG. 1

5. An additional embodiment, FIG. 2; comprises a separate sealed pouch reservoir wherein the diluent is stored within the active ingredient(s) reservoir layer 3 of the device;

6. A method of claim 3 wherein; to allow the manufacture of the device components separately.

7. A method of claim 4 wherein; the mixing of the diluent and/or active ingredient(s) is achieved by rupturing the separate sealed pouch located within the active ingredient reservoir of embodiment 2;

8. A method of both claims 4 and 5 wherein; the diluent and/or active ingredient(s) can be stored separately under different storage conditions of temperature, humidity, etcetera, until time of use;

9. Another aspect of claim 8 wherein; the components can be manufactured separately under separate manufacturing conditions which may be unique to each component;

10. Another aspect of the present invention wherein; the transdermal delivery device will be worn by the patient for predetermined amount of time from 4 to 24 hours or longer;
